(19) United States
(12) Patent Application Publication
(10) Pub. No.: US 2014/0128760 A1
(43) Pub. Date: May 8, 2014

(54) INCONSPICUOUS IMPAIRMENT TESTING DEVICES

(71) Applicant: CAPITAL LOGISTICS, INC., Carson City, NV (US)

(72) Inventor: Barry Saunders, Scottsdale, AZ (US)

(73) Assignee: Capital Logistics, Inc., Carson City, NV (US)

(21) Appl. No.: 13/667,611

(22) Filed: Nov. 2, 2012

(51) Int. Cl. A61B 5/097

(52) U.S. Cl. 600/532

(57) ABSTRACT

An impairment detection device includes a body and a housing removably secured to the body that together resemble a beverage container. A breath analyzer is located within the housing or body and includes a chemical sensor that is configured to detect a chemical impairment level of a user, such as resulting from the use of drugs and/or alcohol. The housing is configured to be removably secured to the body to enable a user to quickly attach and remove the housing from the body. The combination of the housing and body provides a disguise for the impairment detection device because it resembles an actual beverage container. A mouthpiece extends from the housing and is in communication with the chemical sensor for receiving a user breath sample. The housing includes a user interface that is operably associated with the breath analyzer.

### FIELD OF THE INVENTION

The present invention relates generally to testing devices and, more particularly, to devices for detecting chemical impairment of individuals.

### BACKGROUND

Vehicle operation by persons under the influence of alcohol is a well known safety problem in the United States and throughout the world. Thousands of deaths per year in the United States are attributable to drivers operating vehicles under the influence of alcohol. To address this problem, most states have established laws that prohibit operation of a vehicle by an individual with a blood alcohol content (BAC) greater than a preset value (e.g., 0.08% BAC). In addition, “ignition interlock” systems have been developed which are directly connected to a vehicle's ignition system and are designed to prevent vehicle operation by inebriated individuals. Many states require the installation of ignition interlock systems in the vehicles of individuals convicted of driving under the influence of alcohol, particularly repeat offenders.

Conventional ignition interlock systems include a breath analyzer which measures the alcohol content of the breath of an individual. It is well known that the alcohol content of gas present in the alveoli of the lungs has an alcohol content directly proportional to that of the bloodstream of an individual. Blood alcohol content, thus, can be accurately determined by measuring breath alcohol content with a breath analyzer. Typically, in order to start a vehicle equipped with an ignition interlock system, the driver must first blow into the breath analyzer. If the driver's breath alcohol exceeds a preset limit, the vehicle's ignition is disabled and the vehicle is rendered inoperable. If the driver's breath alcohol is below the preset limit, ignition is permitted and the vehicle may be started. Exemplary ignition interlock devices that utilize breath analyzers are described in, for example, U.S. Pat. Nos. 3,780,311; 3,824,537; 3,831,707; and 4,592,443.

Usually, an ignition interlock device is installed to a motor vehicle's dashboard. There is no universal clip for installation so, typically, they are attached with adhesive, hook and loop fasteners, or screws, all of which can modify and “damage” a vehicle dashboard. In general, people do not like having ignition interlock devices installed in their vehicles because of the installation modifications that are required to their dashboards. They are also embarrassed by them. Ignition interlock devices draw attention to themselves by, in some cases, making a car's horn honk or flash lights, but certainly when a person has to use them (blow into them, etc.).

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

According to some embodiments of the present invention, an impairment detection device includes a body and a housing removably secured to the body that together resemble a beverage container (e.g., a cup, tumbler, mug, sports bottle, thermos, etc.). A breath analyzer is located within the housing or body and includes a chemical sensor that is configured to detect a chemical impairment level of a user, such as resulting from the use of drugs and/or alcohol. The housing is configured to be removably secured to the body to enable a user to quickly attach and remove the housing from the body. The combination of the housing and body provides a disguise for the impairment detection device because the combination resembles an actual beverage container.

According to some embodiments of the present invention, the housing is shaped as a top for a beverage container. The housing is configured to be threadingly engaged with the body in some embodiments. In other embodiments, the housing is configured to be removably secured to a container by press-fit or snap-fit. In yet further embodiments, the housing is configured to be pivotably attached to a container, such as via a hinge. A mouthpiece is provided that is in communication with the chemical sensor for receiving a user breath sample. The housing includes a user interface that is operably associated with the breath analyzer. The user interface may include a display, one or more user input controls, and/or at least one speaker.

According to some embodiments of the present invention, the housing is a handle or is shaped to resemble a handle. The handle is configured to be removably secured to the body in various ways. A mouthpiece is provided that is in communication with the chemical sensor for receiving a user breath sample. The handle may include a user interface that is operably associated with a breath analyzer disposed within the handle or within the body. The user interface may include a display and/or at least one user input control.

According to some embodiments of the present invention, the body has an external surface, and the housing is removably secured to the body so as to overlie at least a portion of the external surface. The housing may resemble a calorie counter, timer, or other type of device conventionally attached to beverage containers, particularly sports bottles and containers.

In some embodiments, the chemical sensor is an alcohol sensor that is configured to detect a breath alcohol level of a user. A controller within the housing or body is in communication with the alcohol sensor and is configured to be operably connected to an ignition system of a vehicle. The controller is configured to compare a detected breath alcohol level of a user with a threshold value, and to prevent vehicle ignition if the breath alcohol level detected by the breath analyzer is greater than or equal to the threshold value.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate some exemplary embodiments. The drawings and description together serve to fully explain the exemplary embodiments.

FIG. 1 is a top perspective view of an impairment detection device, according to some embodiments of the present invention, wherein a housing shaped as a top for a beverage container is removably secured to a body, such that the housing and body resemble a beverage container.

FIG. 2 illustrates the impairment detection device of FIG. 1 separated from the body.

FIG. 3A is a block diagram illustrating a breath analyzer within the impairment detection device of FIG. 1, according to some embodiments of the present invention.

FIG. 3B illustrates the breath analyzer of FIG. 3A connected to a vehicle ignition system, according to some embodiments of the present invention.

FIG. 4 illustrates various components of a user interface that may be utilized by an impairment detection device, according to some embodiments of the present invention.

FIG. 5 is a front elevation view of the impairment detection device of FIG. 1.

FIG. 6 illustrates the housing of the impairment detection device of FIG. 5 separated from the body.

FIG. 7 is a side view of the impairment detection device of FIG. 1.

FIG. 8 is a side view of the impairment detection device of FIG. 1 opposite that shown in FIG. 7.

FIG. 9 is a top perspective view of an impairment detection device, according to some embodiments of the present invention, wherein a housing shaped as a top for a beverage container is removably secured to a body, such that the housing and body resemble a beverage container.

FIG. 10 illustrates the housing of the impairment detection device of FIG. 9 separated from the body.

FIG. 11 is a side view of the impairment detection device of FIG. 9.

FIG. 12 is a side elevation view of the impairment detection device of FIG. 9 with the housing separated from the body.

FIG. 13 is top plan view of the impairment detection device of FIG. 9.

FIG. 14A is a top perspective view of an impairment detection device, according to some embodiments of the present invention, wherein a handle is removably secured to a body, such that the handle and body resemble a beverage container.

FIG. 14B illustrates the handle of the impairment detection device of FIG. 14A separated from the body.

FIG. 15 is a top perspective view of an impairment detection device, according to some embodiments of the present invention, wherein a housing is removably secured to a body such that the body and housing together resemble a beverage container.

FIGS. 16 and 17 are exploded views of the impairment detection device of FIG. 15.

FIG. 18 is a top perspective view of an impairment detection device, according to some embodiments of the present invention, wherein a housing is removably secured to a container-shaped body.

FIGS. 19 and 20 are exploded views of the impairment detection device of FIG. 18.

### DETAILED DESCRIPTION

The present invention will now be described more fully hereinafter with reference to the accompanying Figures, in which embodiments of the invention are shown. This invention may, however, be embodied in many different forms and should not be construed as limited to the embodiments set forth herein. Like numbers refer to like elements throughout. In the Figures, certain components or features may be exaggerated for clarity, and broken lines may illustrate optional features or elements unless specified otherwise. Features described with respect to one Figure or embodiment can be associated with another embodiment of the Figures although not specifically described or shown as such.

It will be understood that when a feature or element is referred to as being “on” another feature or element, it can be directly on the other feature or element or intervening features and/or elements may also, be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the Figures. It will be understood that the spatially relative terms are intended to encompass different orientations of the device in use or operation in addition to the orientation depicted in the Figures. For example, if a device in the Figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that although the terms first and second are used herein to describe various features or elements, these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The term “ignition interlock device” refers to a ignition interlock device (e.g., a breath alcohol ignition interlock device, etc.) for a vehicle or other equipment.

The terms “chemical impairment” and “impairment”, as used herein, are interchangeable and refer to the impairment of a person by alcohol or other substances, such as drugs.

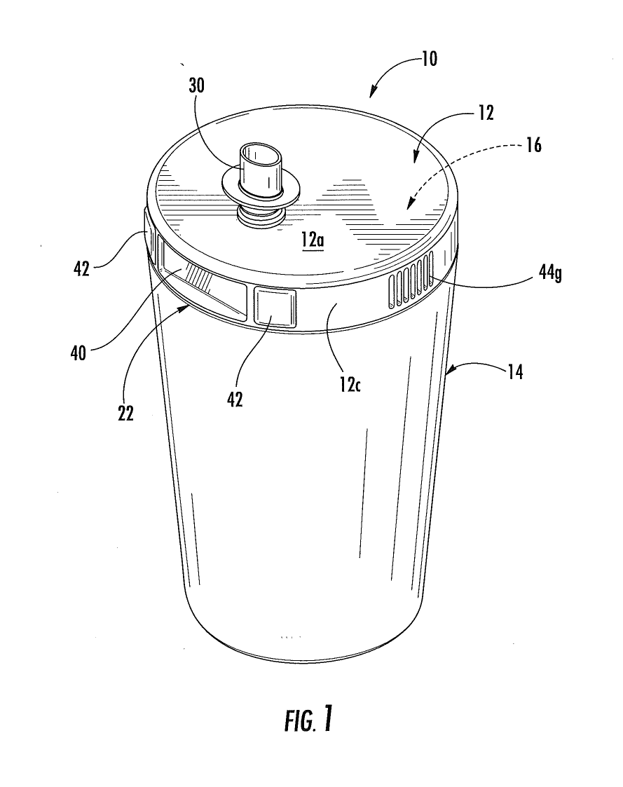

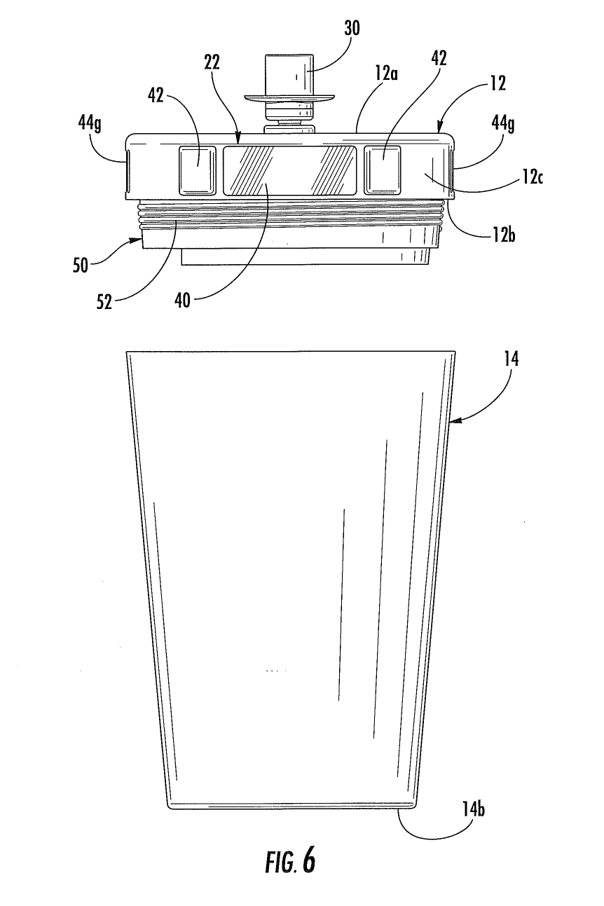

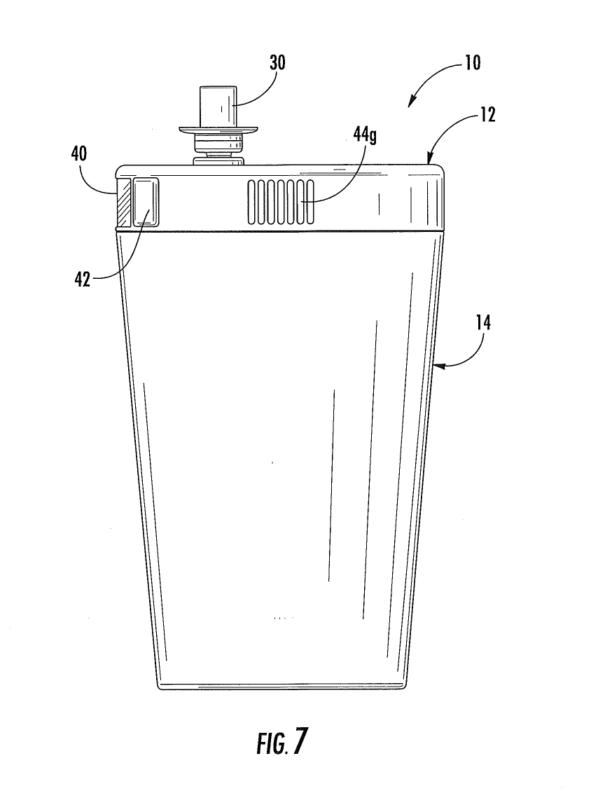

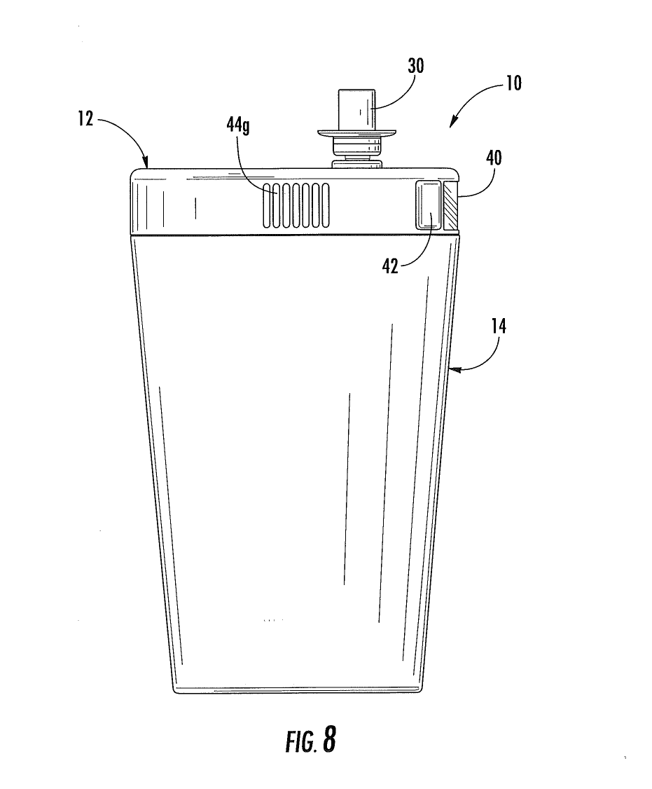

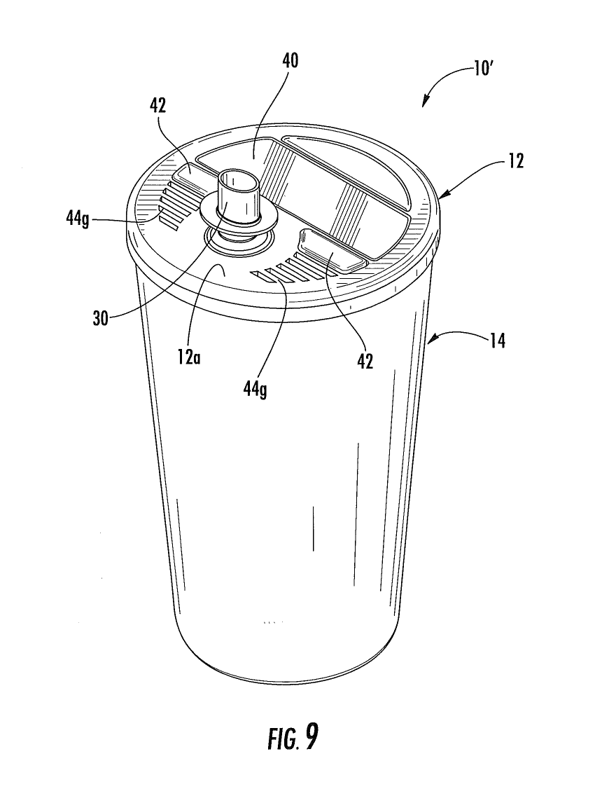

Referring now to FIGS. 1-8, an impairment detection device 10, according to some embodiments of the present invention, is illustrated. The illustrated impairment detection device 10 includes a housing 12 configured to be removably secured to a body 14. The illustrated housing 12 is configured to resemble a beverage container top and together the housing 12 and body 14 resemble a beverage container, such as a cup, tumbler, mug, sports bottle, thermos, etc. The body 14 may have various shapes without limitation. Moreover, the body 14 can have various sizes, colors, and graphics. In addition, the body 14 can have various promotional and/or personalized indicia, including but not limited to team colors and/or insignias of a person's favorite sports team. The terms “indicia” and “promotional indicia” are intended to include all types of printed material including, but not limited to, text, lettering (i.e., alphabetical characters, alphanumeric characters), designs, characters, logos, images, graphics, symbols, etc. Moreover, indicia and promotional indicia may be utilized virtually anywhere on the body 14.

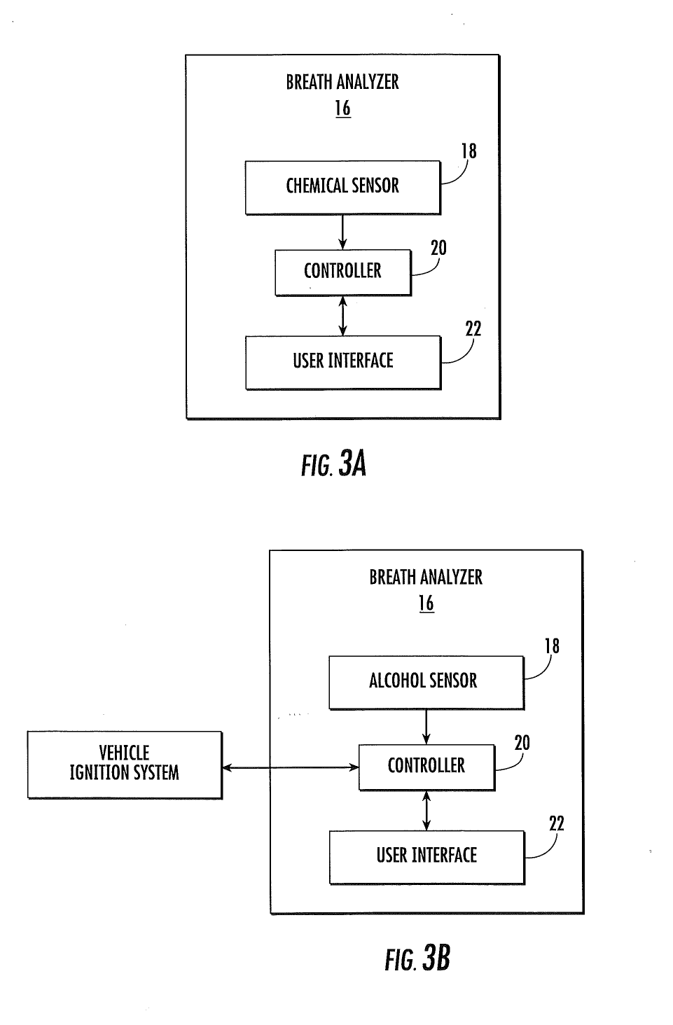

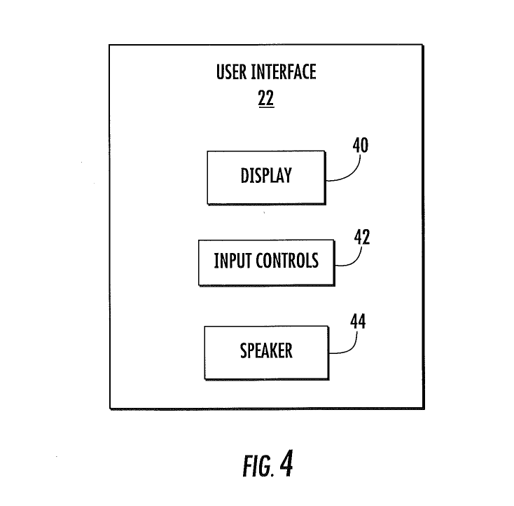

The illustrated impairment detection device 10 includes a breath analyzer 16 (FIG. 3A) located within the housing 12. However, in some embodiments of the present invention, the breath analyzer 16 may be located within the body 14. The breath analyzer 16 includes a chemical sensor 18 (FIG. 3A) that is configured to detect a chemical impairment level of a user. The breath analyzer includes a controller 20 (FIG. 3A) that is in communication with the chemical sensor 18 and that controls operations of the breath analyzer. For example, the controller 20 is configured to compare a detected chemical level of a user with a threshold value and communicate that information to the user. In other embodiments of the present invention, the breath analyzer 16 may be located within the body 14.

In embodiments where the breath analyzer 16 is an alcohol breath analyzer, the controller 20 is configured to compare a detected breath alcohol level of a user with a threshold value and communicate that information to the user. Exemplary types of alcohol sensors that can be used in accordance with embodiments of the present invention include, but are not limited to, electro-chemical fuel cells, infrared sensors, and metal oxide semiconductor sensors. Exemplary fuel cells are available from Draeger USA, Inc., Durango, Colo. Exemplary infrared sensors are available from Cal Sensors, Inc. (e.g., lead selenide and lead sulfide detectors) Santa Rosa, Calif., and Electro Optical Components, Inc., (e.g., pyroelectric and thermopile detectors) Santa Rosa, Calif. Exemplary semiconductor sensors, (e.g., a Taguchi sensor), are available from Figaro USA, Inc., Glenview, Ill.

An individual may be impaired as a result of drug use and/or alcohol use. As such, depending on the type of chemical sensor 18 utilized in the chemical impairment detection device 10, some embodiments of the present invention may be utilized to detect impairment caused by drug use and other embodiments of the present invention may be utilized to detect impairment caused by alcohol use. For example, the chemical sensor 18 may be an alcohol sensor capable of detecting a level of intoxication of an individual based upon a breath sample from the individual and/or a sensor capable of detecting a level of impairment due to drugs and other substances. In some embodiments of the present invention, a combination of sensors that can detect drug use and alcohol use may be utilized in the impairment detection device 10.

A mouthpiece 30 extends from an upper surface 12a of the housing 12 and is in communication with the breath analyzer chemical sensor 18 within the housing 12 (or within the body 14) for receiving a breath sample from a user, such as the driver of a vehicle. The mouthpiece 30 may be configured to resemble a straw extending from the top of a beverage container. However, the mouthpiece 30 can have various shapes and configurations and need not be limited to the shape of a straw, and need not even resemble a straw. Moreover, the mouthpiece 30 need not extend from the housing 12. The mouthpiece 30 may be substantially flush with the upper surface 12a of the housing, for example.

In some embodiments of the present invention, the impairment detection device 10 may serve as a standalone device for determining the impairment of individuals as a result of drug use and/or alcohol use. In some embodiments of the present invention, the impairment detection device 10 may serve as a vehicle ignition interlock device that is connected to the ignition system of a vehicle and designed to prevent vehicle operation by impaired individuals. For example, in some embodiments of the present invention, as illustrated in FIG. 3B, the breath analyzer controller 20 is in communication with an alcohol sensor 18. The controller 20 is configured to be operably connected to an ignition system of a vehicle and is configured to compare a detected breath alcohol level of a user with a threshold value. The controller 20 is configured to prevent vehicle ignition if the breath alcohol level detected by the alcohol sensor 18 is greater than or equal to the threshold value (e.g., by opening a circuit in the vehicle ignition system, etc.). The term “prevent vehicle ignition” is intended to mean preventing a vehicle from being started and/or operated, according to embodiments of the present invention. How a vehicle is prevented from being started and/or operated may be accomplished in any of various ways known to those skilled in the art, and all of such ways are intended to be included within the scope of the present invention.

The controller 20 may be operably associated with various components of a vehicle's ignition and electronics system, which may include, but is not limited to, the battery, vehicle starter switch, tachometer, starter solenoid, etc. The controller 20 may be operably connected to various vehicle systems/components, without limitation, to prevent starting and operation of the vehicle if the vehicle operator fails a breath alcohol test, as would be understood by those skilled in the art. The controller 20 preferably includes a processor and/or other suitable programmable or non-programmable circuitry, including suitable software. The controller 20 may also include other devices as appropriate to control various devices described herein. The controller 20 may be one or more processors or circuitry designed to implement the various functions described herein.

Embodiments of the present invention, when used as vehicle ignition interlock devices, typically are connected to the ignition system of a vehicle via an electrical cord. For example, the breath analyzer controller 20 is connected with the ignition system of a vehicle via an electrical cord. However, in some embodiments of the present invention, the breath analyzer controller 20 may communicate wirelessly with the ignition system of a vehicle. In the illustrated embodiments of the present invention, an electrical cord has been omitted from the figures for ease of illustration. However, one skilled in the art would understand that an electrical cord, when utilized, would extend outwardly from the various impairment detection devices 10, 10′, 100, 200 and 300 and be configured to connect to the ignition system of a vehicle.

In the illustrated embodiment, the breath analyzer 16 includes a user interface 22. The illustrated user interface 22 includes a display 40, user input controls 42, and optionally a speaker 44. The display 40 may be a liquid crystal display (LCD) or any of various types of displays, without limitation. The user interface 22 is configured to display and/or perform operational messages in any of a number of formats via display 40. This includes messages in one or more languages on the display 40 and/or audible operational messages and prompts, including voice messages in one or more languages via the speaker 44. When the impairment detection device 10 is utilized as part of a vehicle ignition interlock system, at various times, instructions and prompts can be provided to a user via the user interface 22, such as regarding when a breath alcohol content retest is required, system status, etc. The user input controls 42 can be utilized by a user to respond to various conditions or requests from the device 10, including the need to enter user identification information, e.g., an entry code number, etc. The illustrated user input controls 42 are push-button type switches. However, various types of user input controls may be utilized without limitation.

Embodiments of the present invention are not limited to a user interface 22 with the illustrated configuration of the display 40, input controls 42, or speaker 44. Various types and configurations of displays may be utilized, without limitation. Moreover, various types, configurations, and numbers of user input controls may be utilized. For example, in some embodiments of the present invention, a touch screen display may be utilized that does not require separate user input controls. More than one speaker 44 may also be utilized. Moreover, a speaker is not required.

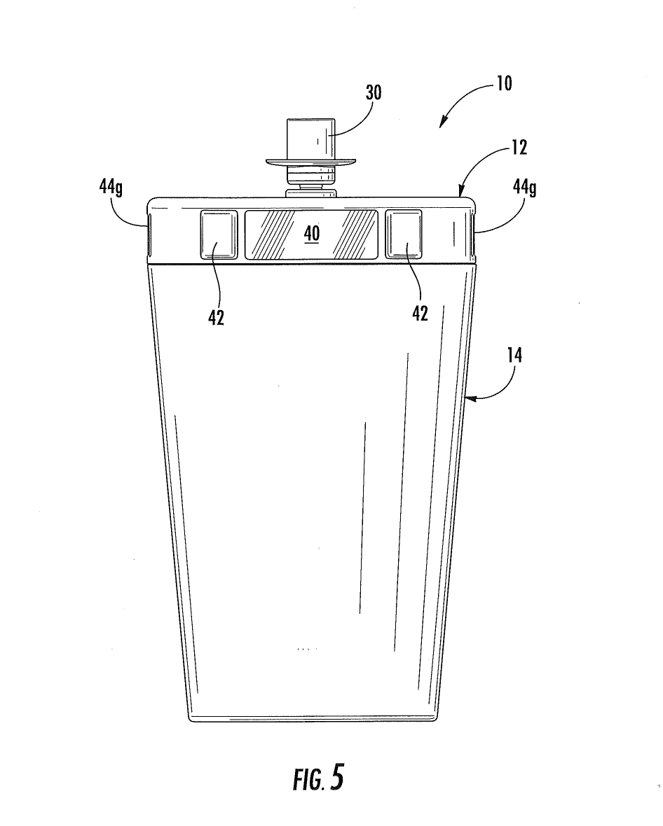

Referring to FIGS. 1, 2 and 5-8, the impairment detection device housing 12 has a disc-like shape of a beverage container top and includes an upper surface 12a, an opposite lower surface 12b, and a peripheral side wall 12c, as illustrated. The display 40 and input controls 42 are located on the housing peripheral side wall 12c, as illustrated. In the illustrated embodiment, a pair of vents or speaker grills 44g are formed through respective portions of the housing peripheral side wall 12c to permit sound from a speaker 44 (FIG. 4) located within the housing 12 to travel outwardly from the housing 12. However embodiments of the present invention do not require vents or grills 44g and do not require a speaker 44 within the housing 12.

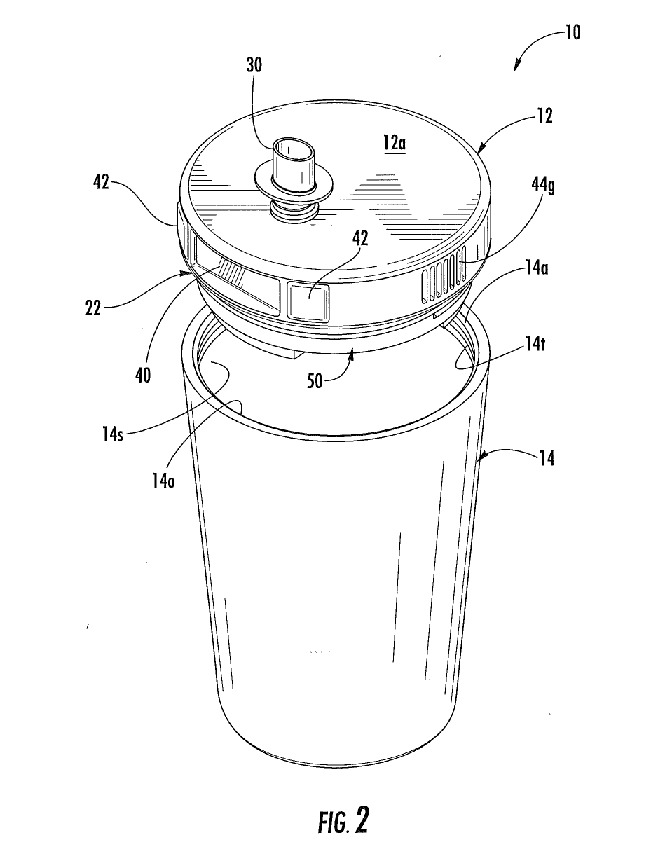

Extending outwardly from the housing lower surface 12b is a connection member 50 that is configured to removably secure the housing 12 to the body 14. In the illustrated embodiment, the connection member 50 includes threads 52 that are configured to threadingly engage corresponding threads 14t in an upper portion 14a of the body 14, as illustrated in FIG. 2. However, embodiments of the present invention are not limited to the housing 12 being threadingly engaged with the body 14. In other embodiments of the present invention, the housing connection member 50 may be shaped and/or configured such that the housing 12 is removably secured to a body 14 by press-fit, snap-fit, etc. In some embodiments, the housing 12 may be pivotably secured to the body 14 via one or more hinges. Various forms of attachment may be utilized that allow the housing 12 to be quickly attached to the body 14 and quickly removed therefrom, without limitation. For example, the housing connection member 50 may have a tapered shape configured to engage the body 14 via the body upper portion opening 14o. As another example, the housing connection member 40 may include one or more detents, each configured to engage with a rib or other member extending circumferentially around inner surface 14s of the body upper portion 14a.

In some embodiments of the present invention, the body 14 may have one or more vents that permit a breath sample provided by a user to be expelled from body 14, as would be understood by those skilled in the art. In some embodiments of the present invention, the body 14 may have an open bottom portion 14b that permits a breath sample to be expelled from the body 14.

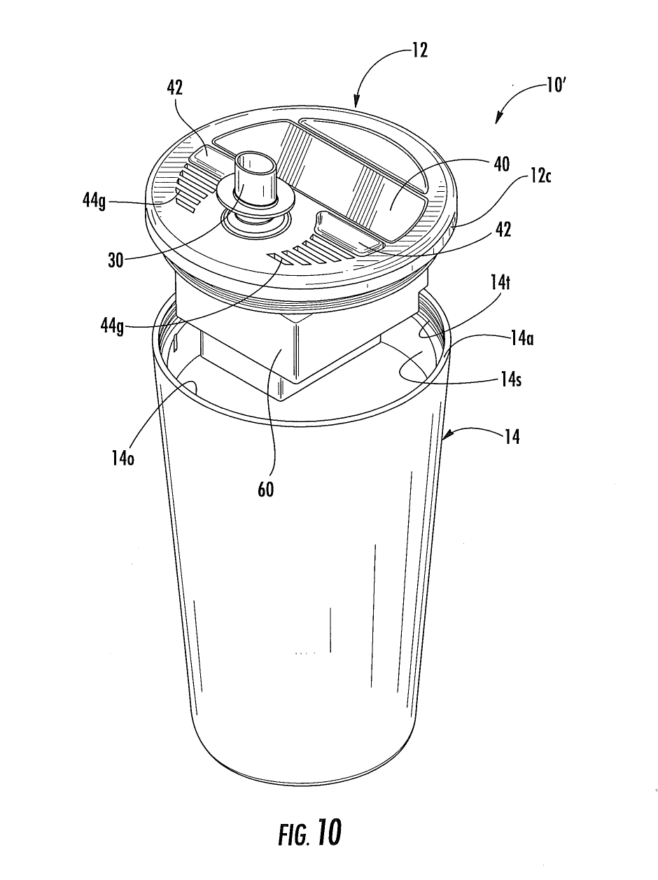

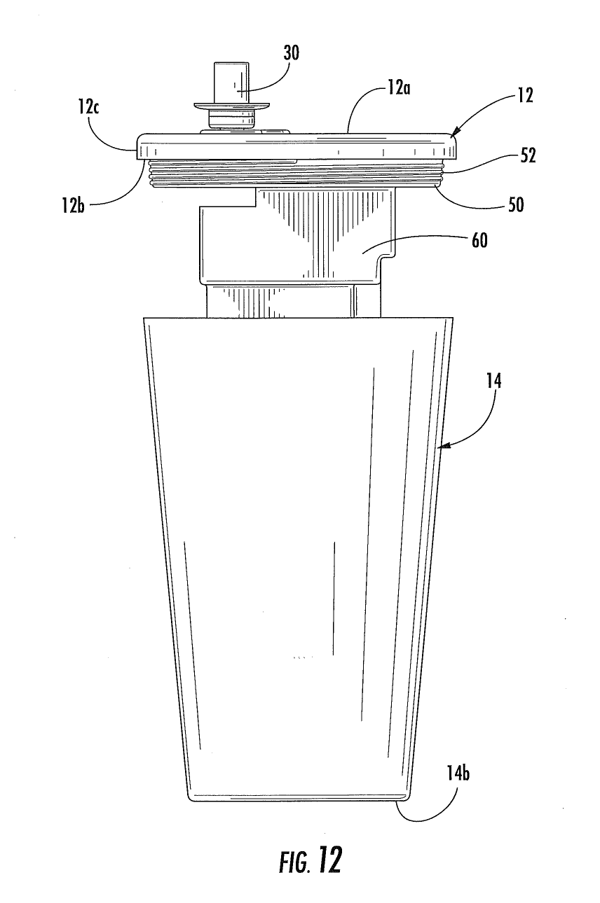

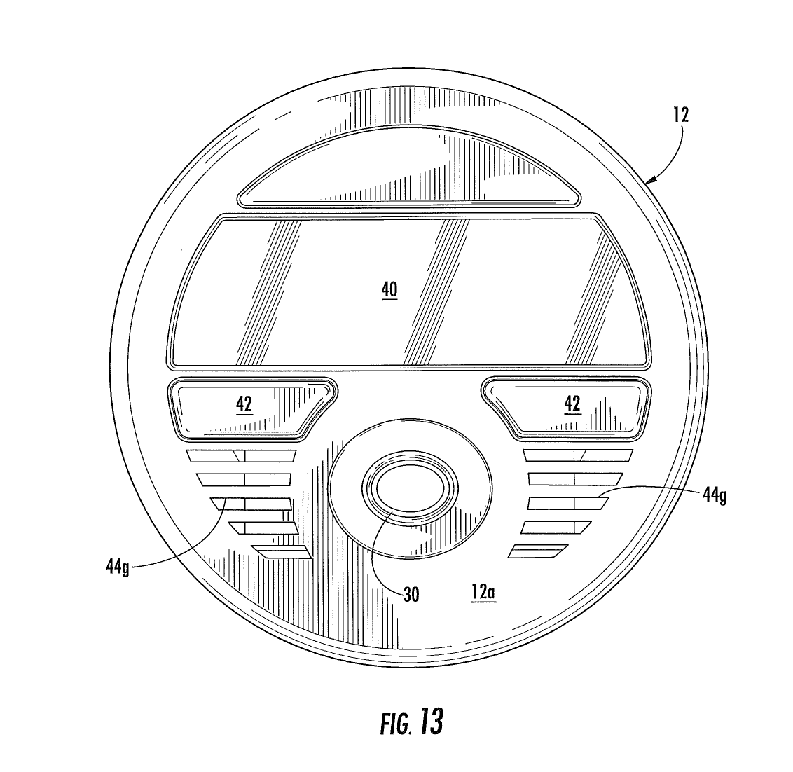

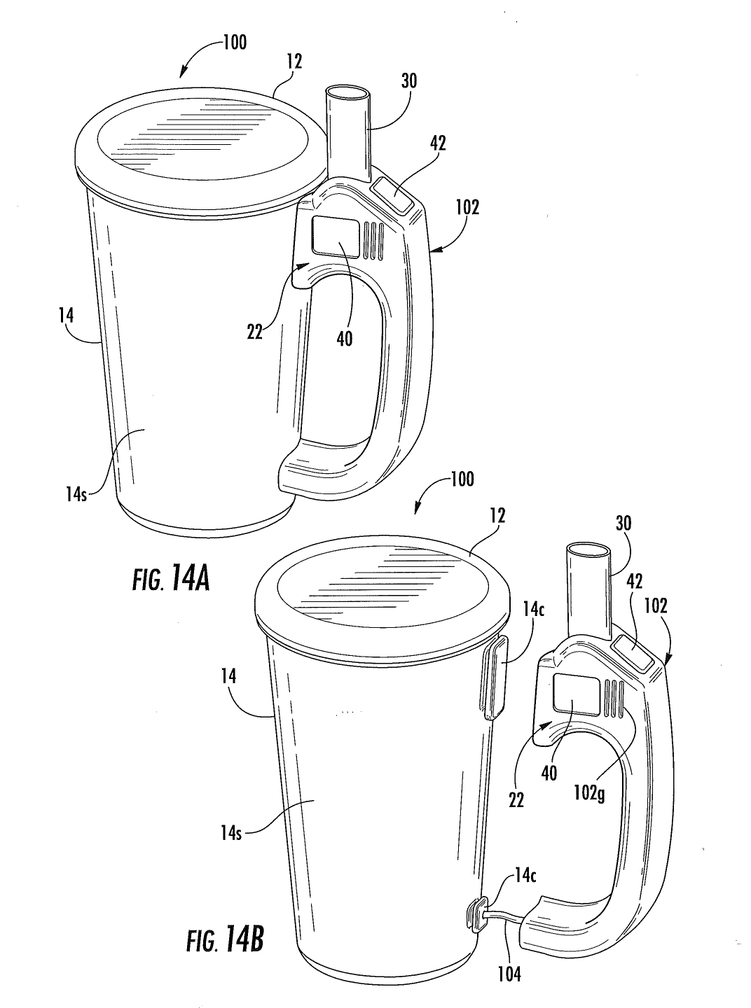

Referring to FIGS. 9-13, an impairment detection device 10′, according to other embodiments of the present invention, is illustrated. The illustrated impairment detection device 10′ includes a housing 12 configured to be removably secured to a body 14. The illustrated housing 12 is configured to resemble a beverage container top and together the housing 12 and body 14 resemble a beverage container. The impairment detection device 10′ includes a breath analyzer 16 (FIG. 3A) located within the housing 12 (or within the body 14). The breath analyzer 16 includes a chemical sensor 18 (FIG. 3A) that is configured to detect a chemical impairment level of a user. The breath analyzer includes a controller 20 that is in communication with the chemical sensor 18 and that controls operations of the breath analyzer. For example, the controller 20 is configured to compare a detected chemical (i.e., drugs or alcohol) level of a user with a threshold value and communicate that information to the user. In embodiments where the breath analyzer 16 is an alcohol breath analyzer, the controller 20 is configured to compare a detected breath alcohol level of a user with a threshold value and communicate that information to the user.

The housing 12 has a disc-like shape of a beverage container top and includes an upper surface 12a, an opposite lower surface 12b, and a peripheral side wall 12c, as illustrated. The display 40 and input controls 42 are located on the housing upper surface 12a, as illustrated. In the illustrated embodiment, a pair of vents or grills 44g are formed through respective portions of the housing upper surface 12a to permit sound from a speaker 44 (FIG. 4) located within the housing 12 to travel outwardly from the housing 12. However, embodiments of the present invention do not require vents or grills 44g and do not require a speaker 44 within the housing 12.

The illustrated impairment detection device 10′ is essentially the same as the device 10 of FIGS. 1, 2 and 5-8 with the exception that the user interface display 40, input controls 42 and grills 44g are located in the upper surface 12a of the housing 12. In addition, the portion of the housing that is exposed when removably secured to the body 14 is relatively thin as compared with the embodiment of FIGS. 1, 2 and 5-8. The illustrated housing 12 in the embodiment of FIGS. 9-13 includes a lower portion 60 that contains the breath analyzer 16. This housing lower portion 60 extends within the container 14 when the housing is removably secured thereto.

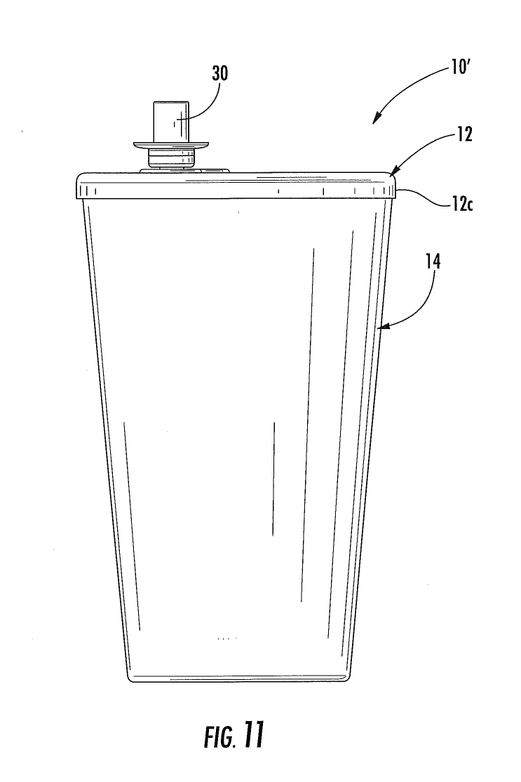

Extending outwardly from the housing lower surface 12b is a connection member 50 that is configured to removably secure the housing 12 to the body 14. In the illustrated embodiment, the connection member 50 includes threads 52 that are configured to threadingly engage corresponding threads 14t in an upper portion 14a of the body 14, as illustrated in FIG. 10. However, embodiments of the present invention are not limited to the housing 12 being threadingly engaged with the body 14. In other embodiments of the present invention, the housing connection member 50 may be shaped and/or configured such that the housing 12 is removably secured to a body 14 by press-fit, snap-fit, hinges, etc. Various forms of attachment may be utilized that allow the housing 12 to be quickly attached to the body 14 and quickly removed therefrom. For example, the housing connection member 50 may have a tapered shape configured to engage the body 14 via the body upper portion opening 14o. As another example, the housing connection member 40 may include one or more detents, each configured to engage with a rib or other member extending circumferentially around inner surface 14s of the container upper portion 14a.

In some embodiments of the present invention, the body 14 may have one or more vents that permit a breath sample provided by a user to be expelled from body 14, as would be understood by those skilled in the art. In some embodiments of the present invention, the body 14 may have an open bottom portion 14b that permits a breath sample to be expelled from the body 14.

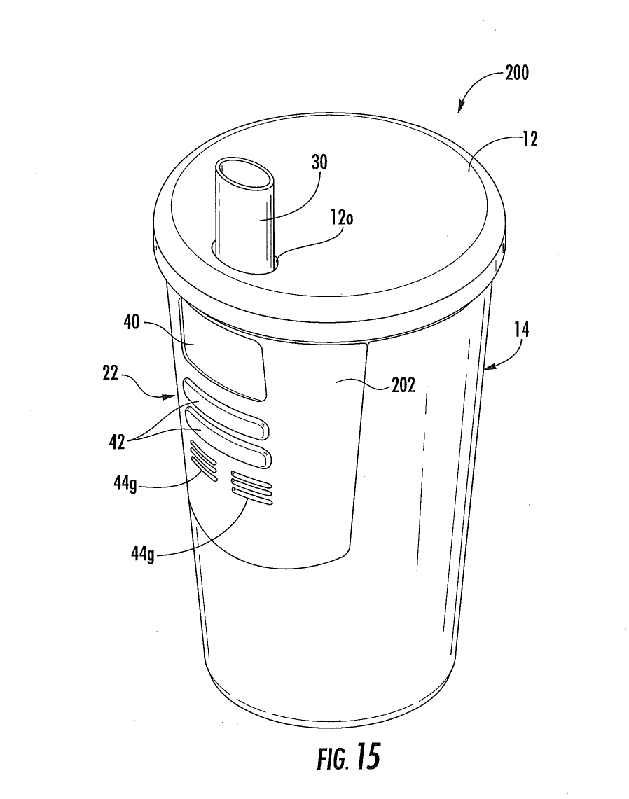

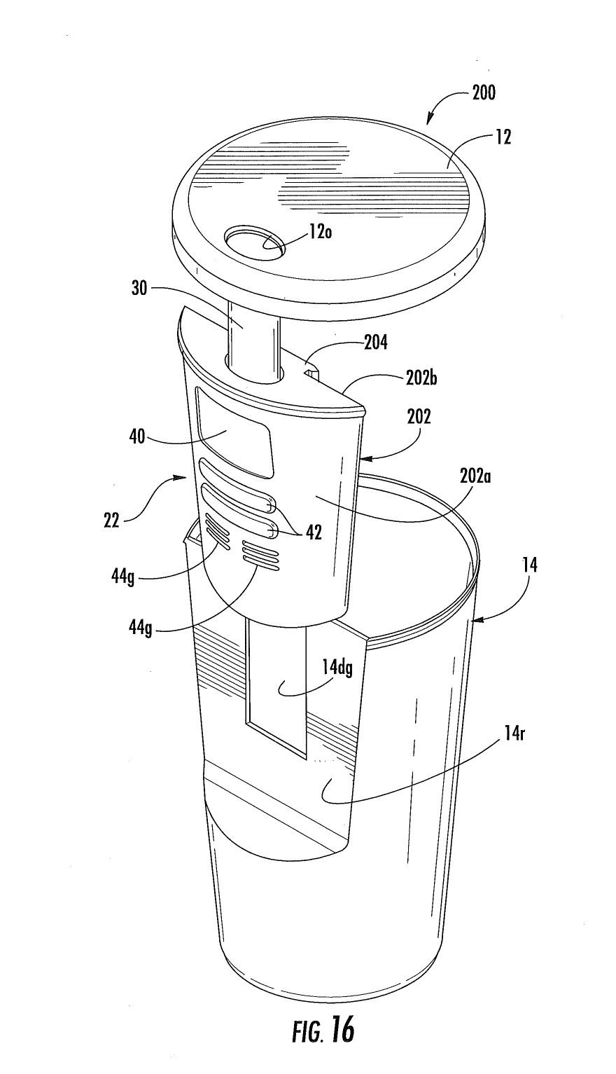

Referring to FIGS. 14A-14B, an impairment detection device 100, according to other embodiments of the present invention, is illustrated. The illustrated impairment detection device 100 includes a handle 102 that is configured to be removably secured to a container-shaped body 14. Together, the handle and container-shaped body 14 resemble a beverage container, such as a mug. The handle 102 may be configured to be removably secured to the body 14 in various ways, without limitation. In the illustrated embodiment, the handle 102 is configured to snap onto the body 14 via connectors 14c extending outwardly from the outer surface 14s of the container-shaped body 14. However, embodiments of the present invention are not limited to the illustrated way of connecting the handle 102 to the container-shaped body 14. In the illustrated embodiment, the container-shaped body 14 includes a lid 12 secured thereto.

The body 14 may have a shape of any type of container including, but not limited to a cup, tumbler, mug, sports bottle, thermos, etc. As described above, the body 14 can have various shapes, sizes, colors, and graphics, and can have various promotional and/or personalized indicia.

A breath analyzer 16 (FIG. 3A) is located within the handle 102 or within the container-shaped body 14. As described above, the breath analyzer 16 includes a chemical sensor 18 (FIG. 3A) that is configured to detect a chemical impairment level of a user. For example, the chemical sensor 18 may be an alcohol sensor capable of detecting a level of intoxication of an individual based upon a breath sample from the individual and/or a sensor capable of detecting a level of impairment due to drugs and other substances. In some embodiments of the present invention, a combination of sensors that can detect drug use and alcohol use may be utilized in the impairment detection device 100.

A mouthpiece 30 extends from the handle 102 and is in communication with the breath analyzer chemical sensor 18 for receiving a breath sample from a user, such as the driver of a vehicle. When the breath analyzer is located within the container-shaped body, the mouthpiece 30 includes additional tubing (not shown) that is configured to maintain the mouthpiece in communication with the breath analyzer chemical sensor 18, as would be understood by those skilled in the art of the present invention. The mouthpiece 30 is configured to resemble a straw extending from the top of a beverage container. However, the mouthpiece 30 can have various shapes and configurations and need not be limited to the shape of a straw, and need not even resemble a straw. Moreover, the mouthpiece 30 need not extend from the handle 102. The mouthpiece 30 may be substantially flush with a surface of the handle 102, for example.

The illustrated handle 102 includes a user interface 22 that is operably associated with the breath analyzer 16. The illustrated user interface 22 includes a display 40 and a user input control 42. An electrical cord 104 extends from the handle to the breath analyzer within the body 14 and connects the user interface 22 to the breath analyzer, as would be understood by those skilled in the art. However, embodiments of the present invention are not limited to a user interface 22 with the illustrated configuration of the display 40 and input control 42. Various types and configurations of displays may be utilized, without limitation. Moreover, various types, configurations, and numbers of user input controls may be utilized. For example, in some embodiments of the present invention, a touch screen display may be utilized that does not require separate user input controls. In the illustrated embodiment, vents or grills 44g are formed through a portion of the handle 102 to permit sound from a speaker 44 (FIG. 4) located within the housing 12 to travel outwardly from the housing 12. However, embodiments of the present invention do not require vents or grills 44g and do not require a speaker 44 within the handle 102.

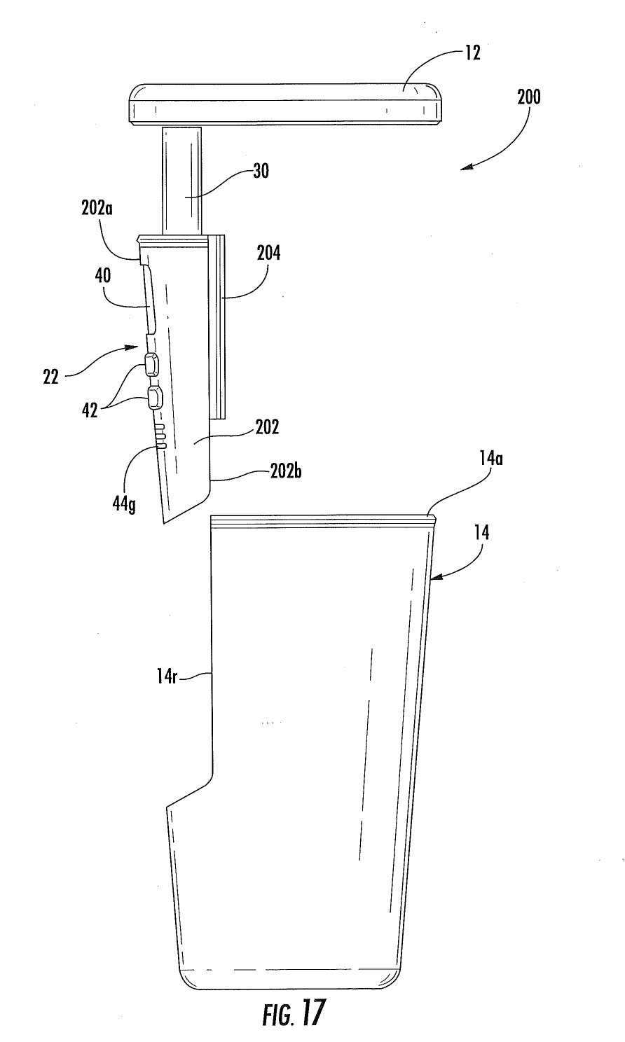

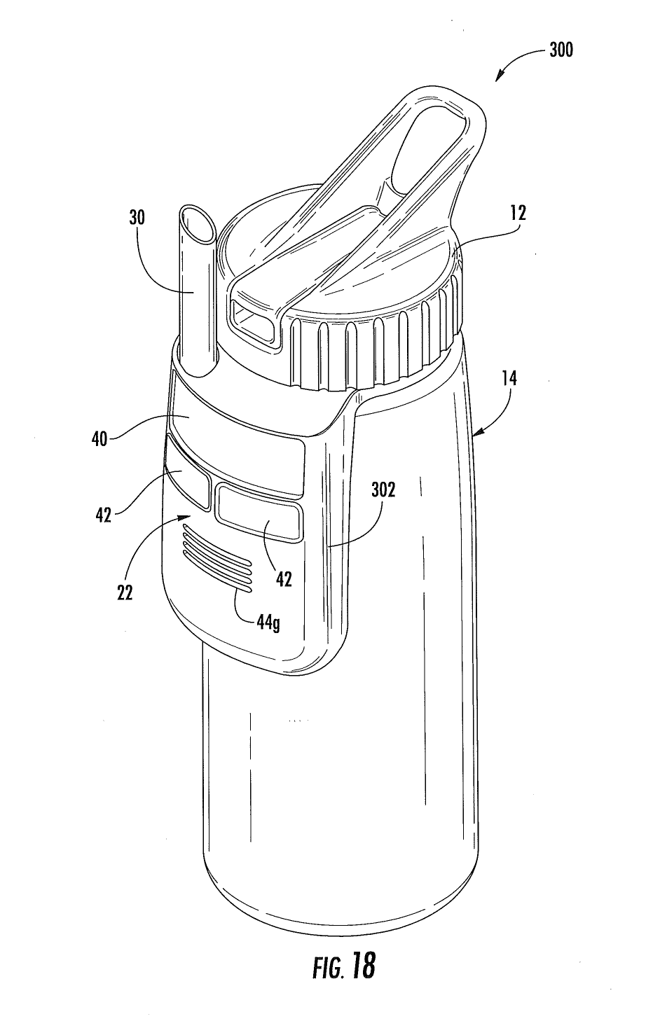

Referring to FIGS. 15-17, an impairment detection device 200, according to other embodiments of the present invention, is illustrated. The illustrated impairment detection device 200 includes a housing 202 that is configured to be removably secured to a body 14, wherein the body 14 and housing 202 together resemble a beverage container. In the illustrated embodiment, the housing 202 has a dovetail-shaped connector 204 extending from a rear surface 202b thereof. The dovetail-shaped connector 204 is configured to slidably engage a corresponding dovetail-shaped groove 14dg formed in a recessed portion 14r of the body 14, as illustrated in FIG. 16. However, the housing 202 may be configured to be removably secured to the body 14 in various ways, without limitation. The illustrated housing 202 has a shape such that, when secured to the body 14, creates the shape of a beverage container. In other words, the housing 202 completes the generally cylindrical or tapered cylindrical configuration of the body 14.

A lid 12 is configured to be removably secured to an upper portion 14a of the body 14 after the housing 202 is secured to the body 14. The lid 12 includes an opening 12o formed therein through which the mouthpiece 30 extends. The lid 12 may be removably secured to the body 14 in various ways without limitation.

As described above, the body 14 may have various shapes, sizes, colors, and graphics. In addition, the body 14 can have various promotional and/or personalized indicia.

A breath analyzer 16 (FIG. 3A) is located within the housing 202. As described above, the breath analyzer 16 includes a chemical sensor 18 (FIG. 3A) that is configured to detect a chemical impairment level of a user. For example, the chemical sensor 18 may be an alcohol sensor capable of detecting a level of intoxication of an individual based upon a breath sample from the individual and/or a sensor capable of detecting a level of impairment due to drugs and other substances. In some embodiments of the present invention, a combination of sensors that can detect drug use and alcohol use may be utilized in the impairment detection device 200.

A mouthpiece 30 extends from the housing 202 and is in communication with the breath analyzer chemical sensor 18 for receiving a breath sample from a user, such as the driver of a vehicle. The mouthpiece 30 is configured to resemble a straw extending from the top of a beverage container. However, the mouthpiece 30 can have various shapes and configurations and need not be limited to the shape of a straw, and need not even resemble a straw. Moreover, the mouthpiece 30 need not extend from the housing 202. The mouthpiece 30 may be substantially flush with a surface of the housing, for example.

The illustrated housing 202 includes a user interface 22 on a front side 202a, thereof, and that is operably associated with the breath analyzer 16. The illustrated user interface 22 includes a display 40 and user input controls 42. However, embodiments of the present invention are not limited to a user interface 22 with the illustrated configuration of the display 40 and input controls 42. Various types and configurations of displays may be utilized, without limitation. Moreover, various types, configurations, and numbers of user input controls may be utilized. For example, in some embodiments of the present invention, a touch screen display may be utilized that does not require separate user input controls. In the illustrated embodiment, vents or grills 44g are formed through a portion of the housing 102 to permit sound from a speaker 44 (FIG. 4) located within the housing 202 to travel outwardly from the housing 202. However, embodiments of the present invention do not require vents or grills 44g and do not require a speaker 44 within the housing 202.

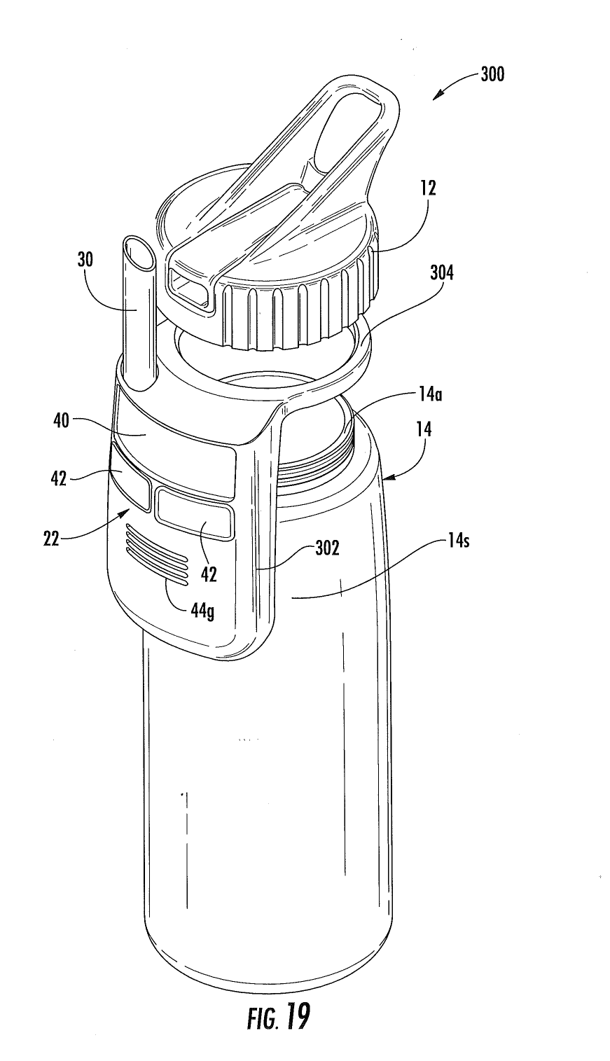

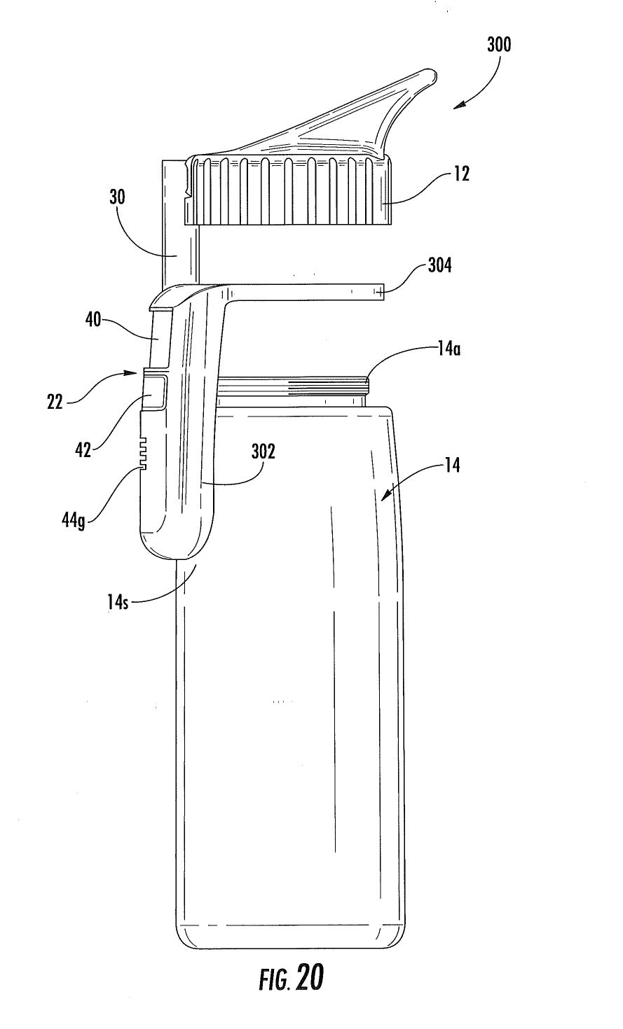

Referring to FIGS. 18-20, an impairment detection device 300, according to other embodiments of the present invention, is illustrated. The illustrated impairment detection device 300 includes a housing 302 that is configured to be removably secured to a body 14. The body 14 is a container, such as a beverage container with an upper portion 14a and an external surface 14s. The housing 302 is removably secured to the body 14 so as to overlie at least a portion of the external surface 14s. The housing 302 resembles a calorie counter, timer, or other device typically associated with a beverage container, such as a sports bottle/container.

The housing 302 includes a ring 304 that is configured to surround the upper portion 14a of the body 14. In the illustrated embodiment, the body upper portion 14a is threaded and a lid 12 is configured to be removably secured to the body upper portion 14a via corresponding threads (not shown) in the lid 12. The lid 12 engages the housing ring 304 and snugly secures the housing 302 to the body 14, as would be understood by those skilled in the art. However, embodiments of the present invention are not limited to the illustrated configuration of the housing 302. The housing 302 may be removably secured to the body 14 in various ways, without limitation.

As described above, the body 14 can have various shapes, sizes, colors, and graphics, and can have various promotional and/or personalized indicia.

A breath analyzer 16 (FIG. 3A) is located within the housing 302. As described above, the breath analyzer 16 includes a chemical sensor 18 (FIG. 3A) that is configured to detect a chemical impairment level of a user. For example, the chemical sensor 18 may be an alcohol sensor capable of detecting a level of intoxication of an individual based upon a breath sample from the individual and/or a sensor capable of detecting a level of impairment due to drugs and other substances. In some embodiments of the present invention, a combination of sensors that can detect drug use and alcohol use may be utilized in the impairment detection device 300.

A mouthpiece 30 extends from the housing 302 and is in communication with the breath analyzer chemical sensor 18 for receiving a breath sample from a user, such as the driver of a vehicle. The mouthpiece 30 is configured to resemble a straw extending from the top of a beverage container. However, the mouthpiece 30 can have various shapes and configurations and need not be limited to the shape of a straw, and need not even resemble a straw. Moreover, the mouthpiece 30 need not extend from the housing 302. The mouthpiece 30 may be substantially flush with the a surface of the housing 302, for example.

The illustrated housing 302 includes a user interface 22 that is operably associated with the breath analyzer 16. The illustrated user interface 22 includes a display 40 and user input controls 42. However, embodiments of the present invention are not limited to a user interface 22 with the illustrated configuration of the display 40 and input controls 42. Various types and configurations of displays may be utilized, without limitation. Moreover, various types, configurations, and numbers of user input controls may be utilized. For example, in some embodiments of the present invention, a touch screen display may be utilized that does not require separate user input controls. In the illustrated embodiment, vents or grills 44g are formed through a portion of the housing 102 to permit sound from a speaker 44 (FIG. 4) located within the housing 302 to travel outwardly from the housing 302. However, embodiments of the present invention do not require vents or grills 44g and do not require a speaker 44 within the housing 302.

In some embodiments of the present invention, the impairment detection devices 10, 10′, 100, 200 and 300 may be configured to operate with a camera or other imaging device provided within a vehicle or other enclosure for verifying that the proper individual is being tested via the impairment detection device 10, 10′, 100, 200 and 300. In some embodiments, the housing 12 of the impairment detection devices 10, 10′, 100, 200 and 300 may include a marking that facilitates the camera to verifying that the proper individual is being tested.

It will be understood that implementations of the present invention are not limited to the specific components disclosed herein, as virtually any components consistent with the intended operation of an inconspicuous impairment detection device implementation may be utilized. Accordingly, for example, although particular components and so forth, are disclosed, such components may comprise any shape, size, style, type, model, version, class, grade, measurement, concentration, material, weight, quantity, and/or the like consistent with the intended operation of an inconspicuous impairment detection device implementation. Implementations are not limited to uses of any specific components, provided that the components selected are consistent with the intended operation of a concealment apparatus implementation.

Accordingly, the components defining any inconspicuous impairment detection device implementation may be formed of any of many different types of materials or combinations thereof that can readily be formed into shaped objects provided that the components selected are consistent with the intended operation of a concealment apparatus implementation. For example, the components may be formed of: rubbers (synthetic and/or natural) and/or other like materials; glasses (such as fiberglass), carbonfiber, aramid-fiber, any combination thereof, and/or other like materials; polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; foams formed of polymers and/or other like materials; composites and/or other like materials; metals, such as zinc, magnesium, titanium, copper, iron, steel, carbon steel, alloy steel, tool steel, stainless steel, spring steel, aluminum, any combination thereof, and/or other like materials; alloys, such as aluminum alloy, titanium alloy, magnesium alloy, copper alloy, any combination thereof, and/or other like materials; any other suitable material; and/or any combination thereof.

For the exemplary purposes of this disclosure, inconspicuous impairment detection device implementations may be made of any material such as polymers, metals, composites, ceramics, and/or the like. The material(s) may be picked so as to make impairment detection devices have any desirable attribute such as strength, light weight, opaqueness, durability, aesthetic appeal, the ability to easily grip and/or hold the impairment detection device, and so forth.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. An impairment detection device, comprising:
a body;
a housing removably secured to the body, wherein the body and housing together resemble a beverage container; and
a breath analyzer within the housing or body, wherein the breath analyzer comprises a chemical sensor configured to detect a chemical impairment level of a user.

2. The device of claim 1, comprising a mouthpiece in communication with the chemical sensor for'receiving a user breath sample.

3. The device of claim 1, wherein the housing comprises a user interface operably associated with the breath analyzer, wherein the user interface comprises a display and/or at least one user input control.

4. The device of claim 1, wherein the housing or body comprises at least one speaker operably associated with the breath analyzer.

5. The device of claim 1, wherein the chemical sensor is an alcohol sensor configured to detect a breath alcohol level of a user.

6. The device of claim 5, further comprising a controller within the housing or body, wherein the controller is in communication with the alcohol sensor, wherein the controller is configured to be operably connected to an ignition system of a vehicle, wherein the controller is configured to compare a detected breath alcohol level of a user with a threshold value, and wherein the controller is configured to prevent vehicle ignition if the breath alcohol level detected by the breath analyzer is greater than or equal to the threshold value.

7. The device of claim 1, wherein the housing is shaped as a beverage container top.

8. The device of claim 7, wherein the housing is threadingly engaged with the body.

9. The device of claim 7, wherein the housing is removably secured to the body by press-fit or snap-fit.

10. The device of claim 1, wherein the housing is a handle.

11. The device of claim 10, comprising a mouthpiece in communication with the chemical sensor for receiving a user breath sample.

12. The device of claim 10, wherein the handle comprises a user interface operably associated with the breath analyzer, wherein the user interface comprises a display and/or at least one user input control.

13. The device of claim 1, wherein the body comprises an external surface, and wherein the housing is removably secured to the body so as to overlie at least a portion of the external surface.

14. An impairment detection device, comprising:
a container-shaped body;
a housing removably secured to the container-shaped body; and
a breath analyzer within the housing or body, wherein the breath analyzer comprises a chemical sensor configured to detect a chemical impairment level of a user.

15. The device of claim 14, comprising a mouthpiece in communication with the chemical sensor for receiving a user breath sample.

16. The device of claim 14, wherein the housing comprises a user interface operably associated with the breath analyzer, wherein the user interface comprises a display and/or at least one user input control.

17. The device of claim 14, wherein the housing comprises at least one speaker operably associated with the breath analyzer.

18. The device of claim 14, wherein the chemical sensor is an alcohol sensor configured to detect a breath alcohol level of a user.

19. The device of claim 18, further comprising a controller within the housing or body, wherein the controller is in communication with the alcohol sensor, wherein the controller is configured to be operably connected to an ignition system of a vehicle, wherein the controller is configured to compare a detected breath alcohol level of a user with a threshold value, and wherein the controller is configured to prevent vehicle ignition if the breath alcohol level detected by the breath analyzer is greater than or equal to the threshold value.

20. The device of claim 14, wherein the housing is shaped as a top for the container-shaped body.

21. The device of claim 20, wherein the housing is configured to be threadingly engaged with the container-shaped body.

22. The device of claim 20, wherein the housing is removably secured to the container-shaped body by press-fit or snap-fit.

23. The device of claim 14, wherein the container-shaped body comprises an external surface, and wherein the housing is removably secured to the container-shaped body so as to overlie at least a portion of the external surface.

24. The device of claim 14, wherein the container-shaped body resembles a beverage container.

25. The device of claim 14, wherein the container-shaped body and housing together resemble a beverage container.

26. The device of claim 14, wherein the housing is a handle.

27. The device of claim 26, comprising a mouthpiece in communication with the chemical sensor for receiving a user breath sample.

28. The device of claim 26, wherein the handle comprises a user interface operably associated with the breath analyzer, wherein the user interface comprises a display and/or at least one user input control.
